(19) United States
(12) Patent Application Publication
(10) Pub. No.: US 2023/0143345 A1
(43) Pub. Date: May 11, 2023

(54) PHARMACEUTICAL COMPOSITION FOR PREVENTION OR TREATMENT OF DISEASES CAUSED BY SARS-COV-2

(71) Applicant: DONG WHA PHARM. CO., LTD., Seoul (KR)

(72) Inventors: Joo Byoung YOON, Hwaseong-si, Gyeonggi-do (KR); Yun Ha HWANG, Gunpo-si Gyeonggi-do (KR); Ma Se LEE, Gyeonggi-do (KR); Hyun Yong LEE, Yongin-si, Gyeonggi-do (KR)

(73) Assignee: DONG WHA PHARM. CO., LTD., Seoul (KR)

(21) Appl. No.: 17/917,905

(22) PCT Filed: Mar. 19, 2021
(86) PCT No.: PCT/KR2021/003462

§ 371 Date: Oct. 7, 2022

(30) Foreign Application Priority Data
Apr. 9, 2020 (KR) 10-2020-0043655
Jul. 20, 2020 (KR) 10-2020-0089909

(51) Int. Cl. A61K 36/19; A61K 31/365; A61P 31/14; A23L 33/105; A23L 33/155; A23L 33/16; A23L 2/68

(52) U.S. Cl. CPC A61K 36/19; A61K 31/365; A61P 31/14; A23L 33/105; A23L 33/155; A23L 33/16; A23L 2/68; A61K 2236/33

(57) ABSTRACT

The present invention relates to a pharmaceutical composition for preventing or treating COVID-19 diseases, comprising an organic solvent extract of Justicia procumbens as an effective ingredient, a pharmaceutical composition for preventing or treating COVID-19 diseases, comprising justicidin-A as an effective ingredient, a pharmaceutical composition for preventing or treating COVID-19 diseases, comprising justicidin-B as an effective ingredient, or a pharmaceutical composition for preventing or treating COVID-19 diseases, comprising 6′ hydroxyl justicidin-B as an effective ingredient, and a food composition thereof for preventing or ameliorating COVID-19 diseases. According to the present invention, an anhydrous ethanol extract of Justicia procumbens, justicidin-A, justicidin-B, and 6′ hydroxyl justicidin-B effectively inhibit SARS-CoV-2 virus, and thus may effectively prevent, treat, or ameliorate diseases caused by SARS-CoV-2 virus.

### TECHNICAL FIELD

The present invention relates to a pharmaceutical composition for preventing or treating diseases caused by SARS-CoV-2, containing an organic solvent extract of Justicia procumbens and active ingredients thereof as an effective ingredient, and a use thereof.

### BACKGROUND ART

SARS-CoV-2 refers to a severe acute respiratory syndrome coronavirus 2, which has been first known in 2019, and is classified as a positive-sense single-stranded RNA virus. The disease infected by this virus has been named Coronavirus disease-19, and is also called COVID-19 for short. The World Health Organization (WHO) has officially announced a coronavirus pandemic. As of December 2020, it has been found that the number of infected people in South Korea is close to 40,000 or more, and about 70 million people or more are infected around the world and this trend continues to spread.

While there is currently no licensed medication to cure COVID-19, numerous clinical trials are under way to find a therapeutic agent for COVID-19, including Kaletra (main ingredient: Lopinavir), which is a therapeutic agent for human immunodeficiency virus (HIV), Remdesivir, which is a therapeutic agent for Ebola virus.

The Justicia genus of the Acanthaceae family consists of about 600 species. Representative plants of the Justicia genus include Justicia procumbens, Justicia pectoralis, Justicia gendarussa, Justicia anselliana, Justicia adhatoda and the like. The plants of the Justicia genus are known to have various physiological activities including an anti-viral effect, but there has not been much research on the active ingredients showing physiological activities with regard to each of those plants. Among those plants, Justicia procumbens is an annual plant that inhabits in Korea, Japan, China, India, and the like. As for the pharmacological activity of Justicia procumbens, the methanol extract of whole plants is known to inhibit the aggregation of platelets and the proliferation of cancer cells in rabbits. It is known that the methanol extract of aerial parts and the lignan-based main constituent ingredients have an effect of inhibiting vesicular stomatitis virus (VSV), human immunodeficiency virus (HIV), etc. (Asano, et al., 1996; XU Xin-Ya, et al., 2019).

However, the effects associated with SARS-CoV-2 have not been elucidated yet.

### DISCLOSURE

### Technical Problem

An object of the present invention is to provide a pharmaceutical composition for preventing or treating diseases caused by SARS-CoV-2, comprising an organic solvent extract of Justicia procumbens as an effective ingredient.

Another object of the present invention is to provide a food composition for preventing or ameliorating diseases caused by SARS-CoV-2, comprising an organic solvent extract of Justicia procumbens as an effective ingredient.

Another object of the present invention is to provide a pharmaceutical composition for preventing or treating diseases caused by SARS-CoV-2, comprising justicidin-A as an effective ingredient.

Another object of the present invention is to provide a food composition for preventing or ameliorating diseases caused by SARS-CoV-2, comprising justicidin-A as an effective ingredient.

Another object of the present invention is to provide a pharmaceutical composition for preventing or treating diseases caused by SARS-CoV-2, comprising justicidin-B as an effective ingredient.

Another object of the present invention is to provide a food composition for preventing or ameliorating diseases caused by SARS-CoV-2, comprising justicidin-B as an effective ingredient.

Another object of the present invention is to provide a pharmaceutical composition for preventing or treating diseases caused by SARS-CoV-2, comprising 6′ hydroxyl justicidin-B as an effective ingredient.

Another object of the present invention is to provide a food composition for preventing or ameliorating diseases caused by SARS-CoV-2, comprising 6′ hydroxyl justicidin-B as an effective ingredient.

### Technical Solution

In order to achieve the above-mentioned object, the present invention provides a pharmaceutical composition for preventing or treating diseases caused by severe acute respiratory syndrome coronavirus 2 (SARS-CoV-2), comprising an extract of Justicia procumbens, justicidin-A, justicidin-B, or 6′ hydroxyl justicidin-B as an effective ingredient.

In addition, the present invention provides an anti-viral composition against

SARS-CoV-2, comprising an extract of Justicia procumbens, justicidin-A, justicidin-B, or 6′ hydroxyl justicidin-B as an effective ingredient.

Moreover, the present invention provides a food composition for preventing or ameliorating diseases caused by SARS-CoV-2, comprising an extract of Justicia procumbens, justicidin-A, justicidin-B, or 6′ hydroxyl justicidin-B as an effective ingredient.

Furthermore, the present invention provides a cosmetic composition for preventing or ameliorating diseases caused by SARS-CoV-2, comprising an extract of Justicia procumbens, justicidin-A, justicidin-B, or 6′ hydroxyl justicidin-B as an effective ingredient.

In the present invention, justicidin-A, justicidin-B, and 6′ hydroxyl justicidin-B includes both the one isolated and purified from Justicia procumbens and chemically synthesized.

In the present invention, the extract of Justicia procumbens, justicidin-A, justicidin-B, and 6′ hydroxyl justicidin-B all inhibit the infection and proliferation of SARS-CoV-2, and thus may effectively prevent, treat, or ameliorate diseases caused by SARS-CoV-2.

In the present invention, the extract of Justicia procumbens may be an organic solvent extract. The organic solvent may include an alcohol having 1 or more and 4 or less carbon atoms.

The organic solvent may include at least one selected from methanol, ethanol, isopropanol, butanol, hexane, ethyl acetate, dichloromethane, ether, chloroform, and acetone. The organic solvent may include, for example, anhydrous ethanol.

The diseases caused by said SARS-CoV-2 may include coronavirus disease-19. Said coronavirus disease-19 may include a respiratory disease. The respiratory disease may include pneumonia. A symptom of said coronavirus disease-19 may include at least one of fever, feebleness, cough, dyspnea, phlegm, sore throat, headache, hemoptysis, nausea, and diarrhea.

The pharmaceutical composition of the present invention may be formulated into a dosage form of tablet, pill, powder, granule, capsule, suspension, liquid for internal use, emulsion, syrup, aerosol, solution for injection, etc., according to a conventional method for preventing and treating diseases caused by SARS-CoV-2. For example, the carriers, excipients, and diluents, which may be contained in the pharmaceutical composition of the present invention, may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. In case of formulating a preparation, the preparation may be prepared by using diluents or excipients such as fillers, extenders, binders, humectants, disintegrants, surfactants, etc., which are generally used.

A solid preparation for oral administration may include tablets, pills, powders, granules, capsules, etc., and this solid preparation may be prepared by mixing at least one excipient, for example, starch, calcium carbonate, sucrose, lactose, gelatin, etc., in a complex composition. In addition, lubricants such as magnesium stearate and talc may be also used in addition to simple excipients. A liquid preparation for oral administration may include suspending agents, liquids for internal use, emulsions, syrups, etc., but may also include various excipients, for example, humectants, sweetening agents, flavoring agents, preservatives, etc. in addition to water and liquid paraffin, which are the frequently used simple diluents.

A preparation for parental administration may include a sterilized aqueous solution, non-aqueous solvent, suspending agent, emulsion, freeze-dried preparation, suppository, etc. The non-aqueous solvent and the suspending agent may include propylene glycol, polyethylene glycol, vegetable oil like olive oil, injectable ester like ethyl oleate, etc.

In addition, the pharmaceutical composition of the present invention may further comprise carriers, excipients, or diluents. The carriers, excipients, or diluents used may include lactose, dextrose, sucrose, sorbitol, mannitol, xylitol, erythritol, maltitol, starch, acacia rubber, alginate, gelatin, calcium phosphate, calcium silicate, cellulose, methyl cellulose, hydroxypropyl methylcellulose, microcrystalline cellulose, polyvinyl pyrrolidone, water, methyl hydroxybenzoate, propyl hydroxybenzoate, talc, magnesium stearate, mineral such as silicon dioxide, etc.

A dosage of the pharmaceutical composition according to the present invention may need to be a pharmaceutically effective amount. The “pharmaceutically effective amount” may mean an amount enough to prevent or treat diseases at a reasonable benefit/risk ratio applicable to medical treatment, and a level of effective dose may be variously selected by those skilled in the art according to factors such as a formulation method, a patient's condition, weight, gender and age, a degree of disease, a drug form, an administration route and period, an excretion rate, reaction sensitivity, etc. The effective amount may vary depending on a route of disposal, a use of excipients and possibility of being used with other drugs, as recognized by those skilled in the art.

According to the present invention, an amount of administering or an amount of taking the organic solvent extract of Justicia procumbens may vary in a range thereof depending on a patient's weight, age, gender, health condition and diet, an administration time, an administration method, an excretion rate, and a severity of disease. However, it is preferable to take 0.001 mg/kg to 1000 mg/kg once or several times by dividing the amount of the extract on an adult basis.

The pharmaceutical composition of the present invention may be administered to mammals such as mice, livestock, humans, etc. through various routes. All the methods of administration may be anticipated, and may be administered, for example, through oral, parenteral, intravenous, intradermic, and subcutaneous injections.

In addition, in another aspect, the present invention provides a method for preventing or treating diseases caused by SARS-CoV-2, including a step of administering a pharmaceutical composition comprising an organic solvent extract of Justicia procumbens into a subject in need thereof.

The present invention also provides a method for preventing or treating diseases caused by SARS-CoV-2, including a step of administering a pharmaceutical composition comprising justicidin-A into a subject in need thereof.

The present invention also provides a method for preventing or treating diseases caused by SARS-CoV-2, including a step of administering a pharmaceutical composition comprising justicidin-B into a subject in need thereof.

The present invention also provides a method for preventing or treating diseases caused by SARS-CoV-2, including a step of administering a pharmaceutical composition comprising 6′ hydroxyl justicidin-B into a subject in need thereof.

In the present invention, the subject refers to animal, and is typically mammal, on which treatment using the drug of the present invention, etc., may exhibit a beneficial effect. A preferable example of this subject may include primates like humans. In addition, those subjects may include all the subjects who have the symptom of diseases caused by SARS-CoV-2 (for example, fever, feebleness, cough, dyspnea, pneumonia, phlegm, sore throat, headache, hemoptysis, nausea, or diarrhea), or may have a risk of developing those symptom.

An amount effective for preventing or treating diseases caused by SARS-CoV-2 refers to an amount capable of achieving a desired outcome in a subject in need of treatment, or an amount capable of providing an objective or subjective advantage to a subject in need of treatment. The amount effective for preventing or treating diseases caused by SARS-CoV-2 may be a single dose or multiple doses. The amount effective for preventing or treating diseases caused by SARS-CoV-2 may include an amount when the drug of the present invention is provided alone, but may also include an amount when the drug of the present invention is provided in combination with one or more other compositions (for example, other therapeutic agents for respiratory disease, etc.).

The numerical values described in the present specification as above should be interpreted to include a range of equivalents thereof, unless otherwise stated.

The present invention also provides a use of the pharmaceutical composition comprising the organic solvent extract of Justicia procumbens as an effective ingredient for preventing or treating diseases caused by SARS-CoV-2.

The present invention also provides a use of the pharmaceutical composition comprising justicidin-A as an effective ingredient for preventing or treating diseases caused by SARS-CoV-2.

The present invention also provides a use of the pharmaceutical composition comprising justicidin-B as an effective ingredient for preventing or treating diseases caused by SARS-CoV-2.

The present invention also provides a use of the pharmaceutical composition comprising 6′ hydroxyl justicidin-B as an effective ingredient for preventing or treating diseases caused by SARS-CoV-2.

The present invention also provides a use of the pharmaceutical composition in the manufacture of a medicament for preventing or treating diseases caused by SARS-CoV-2, wherein the composition comprising the organic solvent extract of Justicia procumbens as an effective ingredient.

The present invention also provides a use of the pharmaceutical composition in the manufacture of a medicament for preventing or treating diseases caused by SARS-CoV-2, wherein the composition comprising justicidin-A as an effective ingredient.

The present invention also provides a use of the pharmaceutical composition in the manufacture of a medicament for preventing or treating diseases caused by SARS-CoV-2, wherein the composition comprising justicidin-B as an effective ingredient.

The present invention also provides a use of the pharmaceutical composition in the manufacture of a medicament for preventing or treating diseases caused by SARS-CoV-2, wherein the composition comprising 6′ hydroxyl justicidin-B as an effective ingredient.

### Advantageous Effects

According to the present invention, each of Justicia procumbens organic solvent extract, justicidin-B, and 6′ hydroxyl justicidin-B shows an effect of inhibiting the intracellular infection and proliferation of SARS-CoV-2 associated with COVID-19 at a higher rate than that of a positive control group, and ultimately can be widely used as an agent for preventing or treating and ameliorating diseases caused by SARS-CoV-2.

### DESCRIPTION OF DRAWINGS

FIG. 1 shows a chemical structure of justicidin-A, justicidin-B, and 6′ hydroxyl justicidin-B according to the present invention.

FIG. 2 shows a position of justicidin-A, justicidin-B, and 6′ hydroxyl justicidin-B from the HPLC results of the anhydrous ethanol extract of Justicia procumbens according to the present invention.

FIG. 3 shows a SARS-CoV-2 virus inhibitory effect and cell viability of Justicia procumbens anhydrous ethanol extract, justicidin-A, justicidin-B, and 6′ hydroxyl justicidin-B according to the present invention.

### MODE FOR INVENTION

Hereinafter, the present invention will be described in detail through preferred Preparing Examples, Examples, and Formulation Examples for better understanding of the present invention. However, the following Preparing Examples, Examples, and Formulation Examples are provided only for the purpose of illustrating the present invention, and thus the present invention is not limited thereto.

### PREPARING EXAMPLE 1

### Preparation of Anhydrous Ethanol Extract of Justicia procumbens

An anhydrous ethanol extract of Justicia procumbens used in an experiment was prepared by using Justicia procumbens (voucher specimen No: NIBRVP0000642884 in 2017) which was cultivated and collected in Jecheon, Chungbuk, and of which origin of the plant has been identified by the National Institute of Biological Resources, Ministry of Environment, Korea.

The dried Justicia procumbens was regularly cut, after which anhydrous ethanol was added 10 times the amount of the Justicia procumbens, immersed and extracted at room temperature for 24 hours, filtered to prepare a first extract, and then concentrated under reduced pressure. Then, anhydrous ethanol was added again eight times the amount of the Justicia procumbens, immersed and extracted again at room temperature for 24 hours, filtered to prepare a second extract, and then concentrated under reduced pressure. The resulting concentrates were combined to prepare an anhydrous ethanol extract of Justicia procumbens having a final solid content by about 10%, and then colloidal silicon dioxide was uniformly mixed so as to be 1:1 compared to the solid content of the extract. The resulting mixture was dried under reduced pressure at 60° C., and then uniformly pulverized to prepare a final anhydrous ethanol extract of Justicia procumbens in powder form.

### Preparing Example 2

### Preparation of Justicidin-A Through an Organic Synthesis Method

Justicidin-A (9-benzo[1,3]dioxol-5-yl-4,6,7-trimethoxy-3H -naphtho[2,3-c]furan-1-one) was prepared through a previously known method (Gunaganti Naresh, et al., 2015) and identified as follows.

¹H-NMR (CDCl₃, 500 MHz) δ 7.54(s, 1H), 7.06(s, 1H), 6.95(d, 1H, J=7.9 Hz), 6.82(d, 1H, J=1.5 Hz), 6.79(dd, 1H, J=1.6, 7.875 Hz), 6.04, 6.08(dd, 2H, J=1.45, 22.775 Hz), 5.54(s, 2H), 4.13(s, 3H), 4.07(s, 3H), 3.80(s, 3H). 13C-NMR (CDCl₃, 125 MHz) δ 169.6, 151.7, 150.4, 147.9, 147.6, ¹47.5, 134.5, 130.7, 128.6, 126.1, 124.6, 123.7, 119.4, 110.9, 108.3, 106.2, 101.3, 100.7, 66.7, 59.7, 56.2, 55.9. The purity of Justicidin-A was 99.52%.

### PREPARING EXAMPLE 3

### Preparation of Justicidin-B Through an Organic Synthesis Method

Justicidin-B (9-benzo[1,3]dioxol-5-yl-6,7-dimethoxy-3H-naphtho[2,3-c]furan one) was prepared through a previously known method (David C. Harrowven, et al., 2001) and identified as follows.

¹H-NMR (CDCl₃, 500 MHz) δ 7.70(s, 1H), 7.18(s, 1H), 7.11(s, 1H), 6.97(d, 1H, J=7.7 Hz), 6.86(d, 1H, J=1.45 Hz), 6.83(dd, 1H, J=1.7, 8.025 Hz), 6.07(d, 2H, J=22.6 Hz), 5.37(s, 2H), 4.05(s, 3H), 3.81(s, 3H). 13C-NMR (CDCl₃, 125 MHz) δ 170.0, 151.9, 150.2, 147.7, 147.6, 139.7, 139.6, 133.3, 128.9, 128.5, 123.6, 118.6, 118.4, 110.7, 108.3, 106.1, 105.9, 101.3, 68.1, 56.2, 55.9. The purity of Justicidin-B was 98.55%.

### PREPARING EXAMPLE 4

### Preparation of 6′ Hydroxyl Justicidin-B Through an Organic Synthesis Method

6′ hydroxyl justicidin-B was synthesized through the following process.

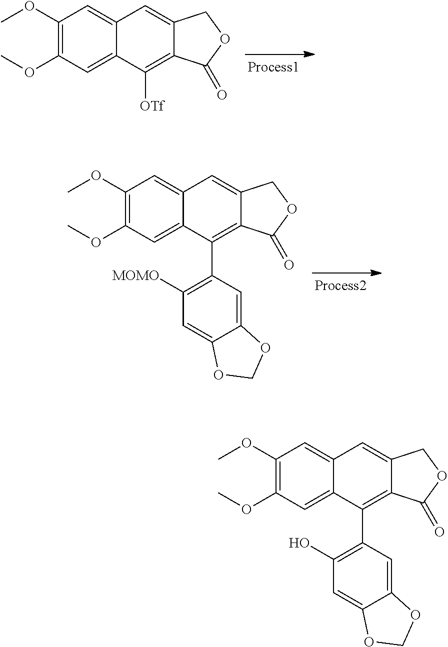

Process 1: 6,7-dimethoxy-3-oxo-1,3-dihydronaphtho[2,3-c]furan-4-yl trifluoromethanesulfonate (3.0 g, 7.65 mmol) was dissolved in dioxane solvent (90 ml), and then 5-methoxymethoxy-6-(4,4,5,5-tetramethyl-[1,3,2]dioxaborolan-2-yl)-benzo[1,3]dioxol (2.83 g, 9.18 mmol), [1,1′-bis(diphenylphosphino)ferrocene]dichloropalladium (II) (1.12 g, 1.53 mmol), lithium hydroxide monohydrate (642 mg, 15.3 mmol) were subsequently added under a nitrogen atmosphere. A temperature was raised up to 60° C. and reacted for four hours. After cooling down to room temperature, water was added to terminate the reaction, and then an extraction was performed with dichloromethane. An organic layer was dried over anhydrous Na₂SO₄, filtered and concentrated. A residue was purified by silica gel column chromatography so as to obtain the title compound of 6,7-dimethoxy-9-(6-methoxymethoxy-benzo[1,3]dioxol-5-yl)-3H-naphtho[2,3-c]furan-1-one (9 g, 21.2 mmol).

¹H NMR: (DMSO-d₆, 400 MHz) 87.92 (s, 1H), 7.48 (s, 1H), 6.95 (s, 1H), 6.87 (s, 1H), 6.73 (s, 1H), 6.08 (d, 2H, J=2.8 Hz), 5.38-5.49 (m, 2H), 4.90 (d, 1H, J=6.8 Hz), 4.81 (d, 1H, J=6.8 Hz), 3.93 (s, 3H), 3.65 (s, 3H), 2.93 (s, 3H).

Process 2: 6,7-dimethoxy-9-(6-methoxymethoxy-benzo[1,3]dioxol-5-yl)-3H-naphtho[2,3-c]furan-1-one (8 g, 18.9 mmol) was dissolved in ethanol (80 ml), and 44 ml of 12M HCl was added dropwise thereto, and the above reacted at 50° C. for 12 hours. After the reaction was completed, 100 ml of water was added, and then an extraction was performed with dichloromethane (200 ml, 100 ml, 50 ml). An obtained organic layer was washed with water (150 ml) and brine (100 ml), dried over anhydrous Na₂SO₄, filtered and concentrated. A residue was purified by silica gel column chromatography so as to obtain the title compound of 9-(6-hydroxy-benzo[1,3]dioxol-5-yl)-6,7-dimethoxy-3H-naphtho[2,3-c]furan-1-one (6′-HJB, 5.20 g, 13.7 mmol, 72.5%). The purity of 6′ hydroxyl justicidin-B was 98.76%.

¹H NMR: (DMSO-d₆, 400 MHz) δ 9.01 (s, 1H), 7.88 (s, 1H), 7.46 (s, 1H), 6.95 (s, 1H), 6.63 (s, 1H), 6.57 (s, 1H), 6.00 (d, 2H, J=2.00 Hz), 5.41 (s, 2H), 3.93 (s, 3H), 3.66 (s, 3H).

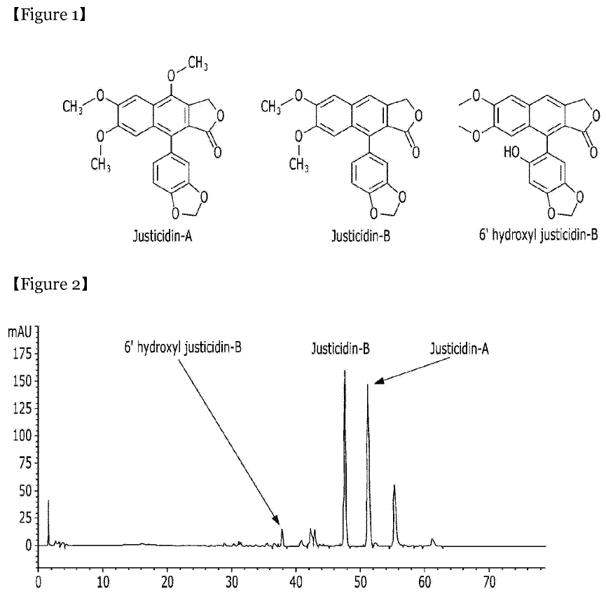

A structure of justicidin-A, justicidin-B, and 6′ hydroxyl justicidin-B are shown in FIG. 1.

### PREPARING EXAMPLE 5

### Preparation of Hydrous Ethanol Extract and Organic Solvent Extract of Justicia procumbens

An anhydrous ethanol extract of Justicia procumbens used in an experiment was prepared by using Justicia procumbens (voucher specimen No: NIBRVP0000590019 in 2016) which was cultivated and collected in Jecheon, Chungbuk, and of which origin of the plant has been identified by the National Institute of Biological Resources, Ministry of Environment, Korea.

After the dried Justicia procumbens was regularly cut and pulverized, which 10 g thereof was accurately taken and subjected to an extraction twice (two hours for a first extraction and one hour for a second extraction) in an amount of 100 ml under refluxing with ethanol, 95% hydrous ethanol, 60% hydrous ethanol, 30% hydrous ethanol, water, butanol, acetone, ethyl acetate, hexane, isopropanol, methanol, and dichloromethane as a solvent at 80 to 90° C., filtered, and concentrated under reduced pressure so as to obtain 0.2 to 1.9 g of the title compound (See Table 1).

TABLE 1

| Extraction solvent | Obtained amount (g) | Yield rate (%) |
|---|---|---|
| Ethanol | 0.5 | 4.9 |
| 95% hydrous | 0.8 | 8.1 |
| ethanol | | |
| 60% hydrous | 1.5 | 15.1 |
| ethanol | | |
| 30% hydrous | 1.8 | 18.4 |
| ethanol | | |
| Water | 1.9 | 19.4 |
| Butanol | 0.4 | 4.1 |
| Acetone | 0.2 | 2.4 |
| Ethyl acetate | 0.3 | 3.3 |
| Hexane | 0.2 | 1.5 |
| Isopropanol | 0.4 | 3.6 |
| Methanol | 0.9 | 9.0 |
| Dichloromethane | 0.3 | 2.6 |

### EXAMPLE 1

### Pattern of High Speed Liquid Chromatography (HPLC) of Extract of Justicia procumbens and Identification of Justicidin-A, Justicidin-B, and 6′ Hydroxyl Justicidin-B

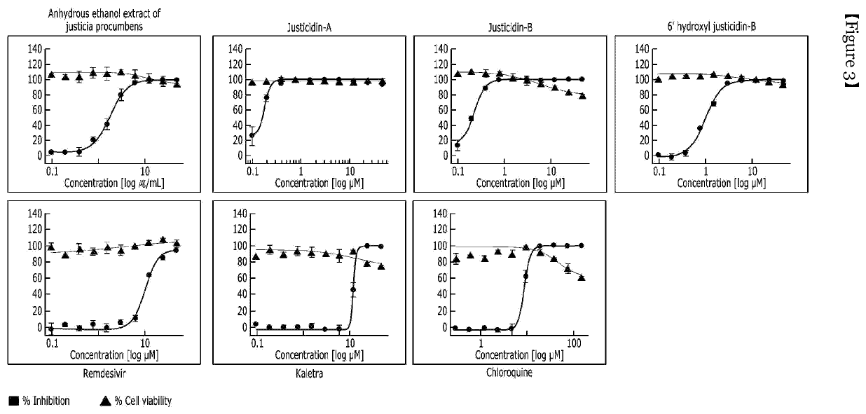

High-speed liquid chromatography (HPLC, Agilent 1260, USA) was performed under the conditions as shown in Table 2 below in order to identifyan active ingredient included in the anhydrous ethanol extract of Justicia procumbens prepared by the preparation method of above Preparing Example 1, and the results are shown in FIG. 2.

TABLE 2

| Detector | Ultraviolet absorption spectrophotometer | | |
|---|---|---|---|
| Detection wavelength | UV 256 nm | | |
| Column | Capcellpak C₁₈ UG120 (4.6 × 250 mm, 5 μm) | | |
| Column temperature | 35° C. | | |
| | <Gradient program> | | |
| | Time | | % |
| | (minute) | % Acetonitrile | Water |
| Mobile phase | 0 | 15 | 85 |
| | 5 | 15 | 85 |
| | 40 | 46 | 54 |
| | 60 | 55 | 45 |
| | 70 | 60 | 40 |
| | 75 | 40 | 60 |
| | 76 | 15 | 85 |

| | |
|---|---|
| Flux | 0.8 mL/min |
| Injection amount | 10 μl |

As a result of the HPLC test, justicidin-A, justicidin-B, and 6′ hydroxyl justicidin-B were detected from the anhydrous ethanol extract of Justicia procumbens. Specifically, the peak of justicidin-A was detected from the anhydrous ethanol extract of Justicia procumbens at a retention time (RT) of about 52 minutes, the peak of justicidin-B was detected at the RT of about 48 minutes, and the peak of 6′ hydroxyl justicidin-B was detected at the RT of about 38 minutes.

### EXAMPLE 2

### Experiment on Comparison of Inhibitory Effects In Vero Cell Lines Infected with SARS-CoV-2

To confirm a SARS-CoV-2 inhibitory effect of an anhydrous ethanol immersion extract of Justicia procumbens, main ingredients thereof such as justicidin-A, justicidin-B, and 6′ hydroxyl justicidin-B, a hydrous ethanol reflux extract of Justicia procumbens, and various organic solvent extracts of Justicia procumbens, the Vero cell lines were cultured for 24 hours, and dosed with the test drugs prepared in Preparing Examples 1 to 5 at 10 concentrations ranging from 0.1 micromolar (or microgram) to 50 micromolar (or microgram), while being infected with SARS-CoV-2 provided by the Korea Centers for Disease Control and Prevention (KCDC). Particularly, the Vero cells were seeded at 1.2×10⁴ per well into a 384-well tissue culture plate. On the following day, the compound at each concentration was subjected to a two-fold serial dilution and the Vero cells were treated therewith. Soon after, the cells treated with the compound were cultured at 37° C. for 24 hours to be infected with SARS-CoV-2 (COVID 19). Then, the cells were fixed, after which the cell membrane was subjected to permeabilization and treated with a primary antibody of anti-SARS-CoV-2 Nucleocapsid (N), and then also treated with a secondary antibody at 37° C. Cell nuclei were identified by staining with Hoechst 33342, and a fluorescence expression was subjected to imaging by using an Operetta high-content image analyzer (Perkin Elmer). Based on the resulting images, the proportion of infected cells was calculated and SARS-CoV-2 virus inhibitory efficacy of the drug was measured with Image Mining (IM) software, an internal analysis program. The SARS-CoV-2 virus inhibitory ability of drugs was summarized in Tables 3 to 5. In addition, the response curve and 50% inhibitory ability (IC₅₀) values *?*according to a drug concentration were derived by using XLFit (IDBS) software, and the results were summarized in Tables 6 to 7 and FIG. 3. The cell viability for each of the test materials is shown in Tables 8 to 10 and FIG. 3.

TABLE 3

| | SARS-CoV-2 virus inhibitory ability (%, average ± standard deviation) | | | | | |
|---|---|---|---|---|---|---|
| | Anhydrous | | | | | |
| | ethanol | | | | | |
| | immersion | | | | | Chloroquine |
| | extract of | | | Remdesivir | Lopinavir | (uM) |
| Con- | | | 6′ hydroxyl | (uM) | (uM) | Positive |
| cent | | Justicidin-B | justicidin-B | Positive | Positive | control |
| ration | (ug/mL) | (uM) | (uM) | control group | control group | group |
| 50 | 99.19 ± 0.26 | 100.28 ± 0.49 | 97.75 ± 2.47 | 94.38 ± 0.41 | 98.23 ± 2.11 | 99.37 ± 1.00 |
| 25 | 99.46 ± 0.11 | 100.00 ± 0.21 | 99.79 ± 0.60 | 84.62 ± 2.21 | 99.01 ± 0.02 | 98.93 ± 0.07 |
| 12.5 | 99.09 ± 0.82 | 100.01 ± 0.14 | 98.87 ± 1.49 | 63.58 ± 1.23 | 45.51 ± 8.72 | 99.31 ± 0.39 |
| 6.25 | 96.26 ± 2.78 | 98:73 ± 2.07 | 99.74 ± 0.24 | 10.79 ± 43.80 | 3.38 ± 5.94 | 97.70 ± 0.16 |
| 3.13 | 80.13 ± 7.59 | 100.03 ± 0.27 | 95.86 ± 0.86 | 5.89 ± 3.28 | 3.03 ± 0.67 | 61.07 ± 7.72 |
| 1.56 | 41.41 ± 7.30 | 99.61 ± 0.65 | 68.58 ± 3.12 | 0.65 ± 5.05 | 0.79 ± 3.76 | 3.52 ± 6.42 |
| 0.78 | 20.58 ± 3.88 | 99.26 ± 0.41 | 35.60 ± 0.69 | 3.03 ± 5.01 | 0.30 ± 2.05 | −4.90 ± 2.07 |
| 0.39 | 4.84 ± 5.63 | 88.06 ± 0.59 | 3.39 ± 3.95 | −1.29 ± 2.86 | −0.60 ± 2.75 | −2.86 ± 0.21 |
| 0.20 | 5.29 ± 0.24 | 48.73 ± 2.82 | −1.71 ± 4.34 | 2.60 ± 1.90 | −0.59 ± 1.18 | −4.26 ± 0.03 |
| 0.10 | 4.46 ± 2.11 | 13.37 ± 6.72 | 1.45 ± 1.16 | −2.24 ± 7.45 | 3.14 ± 0.24 | −2.83 ± 2.09 |

TABLE 4

| | SARS-CoV-2 virus inhibitory ability (%, average ± standard deviation) | | | | | | |
|---|---|---|---|---|---|---|---|
| | | | 30% | 60% | 95% | 100% | |
| | | Water | ethanol | ethanol | ethanol | ethanol | Lopinavir |
| | | extract of | extract of | extract of | extract of | extract of | (uM) |
| Con- | | Justicia | Justicia | Justicia | Justicia | Justicia | Positive |
| cent- | Justicidin-A | procumbens | procumbens | procumbens | procumbens | procumbens | control |
| ration | (uM) | (ug/mL.) | (ug/mL) | (ug/mL) | (ug/mL) | (ug/mL) | group |
| 50 | 93.3 ± 1.69 | 18.7 ± 2.84 | 98.9 ± 1.07 | 99.5 ± 0.16 | 99.6 ± 0.32 | 99.4 ± 0.35 | 99.6 ± 0.57 |
| 25 | 95.3 ± 0.61 | 10.5 ± 1.66 | 98.9 ± 0.88 | 99.1 ± 0.30 | 99.9 ± 0.35 | 99.3 ± 0.01 | 98.6 ± 0.28 |
| 12.5 | 98.1 ± 1.47 | 6.9 ± 3.00 | 96.5 ± 0.42 | 97.0 ± 0.73 | 99.4 ± 6.08 | 99.1 ± 0.26 | 22.6 ± 4.07 |
| 6.25 | 99.6 ± 0.16 | 7.2 ± 2.92 | 79.1 ± 1.91 | 90.3 ± 6.96 | 98.8 ± 0.45 | 99.1 ± 0.61 | −0.1 ± 1.27 |
| 3.13 | 100.0 ± 0.36 | 0.7 ± 0.20 | 22.2 ± 5.15 | 73.4 ± 0.53 | 93.7 ± 2.41 | 98.5 ± 0.25 | 0.2 ± 6.78 |
| 1.56 | 99.0 ± 0.57 | 4.1 ± 5.49 | 8.0 ± 1.92 | 34.2 ± 6.43 | 58.5 ± 21.8 | 70.7 ± 7.31 | 1.7 ± 0.27 |
| 0.78 | 99.0 ± 1.58 | 1.5 ± 8.31 | 13.4 ± 6.95 | 26.4 ± 1.59 | 29.0 ± 0.74 | 53.9 ± 16.87 | 3.1 ± 3.84 |
| 0.39 | 95.2 ± 1.48 | 3.4 ± 8.96 | 7.2 ± 7.92 | 24.5 ± 6.57 | 22.0 ± 3.12 | 44.8 ± 4.59 | 1.2 ± 5.71 |
| 0.20 | 75.3 ± 4.37 | 3.4 ± 0.66 | 8.5 ± 6.36 | 14.8 ± 2.64 | 15.1 ± 0.80 | 23.2 ± 1.74 | 0.2 ± 2.44 |
| 0.10 | 25.7 ± 12.33 | 1.3 ± 2.74 | 12.1 ± 16.70 | 9.9 ± 8.26 | 11.2 ± 6.96 | 13.4 ± 10.56 | −0.2 ± 4.23 |

TABLE 5

| | SARS-CoV-2 virus inhibitory ability (%, average ± standard deviation) | | | | | | |
|---|---|---|---|---|---|---|---|
| | | | | | Dichlorome- | Ethyl | |
| | Isopropanol | Methanol | Butanol | Acetone | thane | acetate | Remdesivir |
| | extract of | extract of | extractor | extract of | extract of | extract of | (uM)- |
| Con- | Justicia | Justicia | Justicia | Justicia | Justicia | Justicia | Positive |
| cent- | procumbens | procumbens | procumbens | procumbens | procumbens | procumbens | control |
| ration | (ug/mL) | (ug/mL) | (ug/mL) | (ug/mL) | (ug/mL) | (ug/mL) | group |
| 50 | 99.0 ± 0.67 | 99.0 ± 0.39 | 99.3 ± 0.45 | 99.0 ± 0.05 | 99:2 ± 0.30 | 99.4 ± 0.27 | 93.5 ± 1.80 |
| 25 | 99.5 ± 0.07 | 99.4 ± 0.39 | 99.0 ± 0.03 | 99.4 ± 0.19 | 99.4 ± 0.28 | 98.9 ± 0.04 | 82.1 ± 1.40 |
| 12.5 | 99.3 ± 0.25 | 99.5 ± 0.06 | 99.6 ± 0.06 | 99.7 ± 0.04 | 99.6 ± 0.17 | 99.4 ± 0:08 | 47.9 ± 0.01 |
| 6.25 | 99.3 ± 0.86 | 96.7 ± 2.87 | 99.0 ± 0.71 | 99.3 ± 0.09 | 99.3 ± 0.41 | 99.4 ± 0.14 | 16.2 ± 2.56 |
| 3.13 | 98.8 ± 0.29 | 88.6 ± 1.65 | 98.9 ± 0.89 | 99.6 ± 0.28 | 98.9 ± 0:69 | 99.3 ± 0.02 | 11.8 ± 0.80 |
| 1.56 | 95.7 ± 0.53 | 54.7 ± 0.63 | 93.3 ± 2.76 | 99.0 ± 8.64 | 95.8 ± 1.53 | 95.4 ± 1.17 | 7.4 ± 1.96 |
| 0.78 | 67.1 ± 6.57 | 41.7 ± 10.09 | 78.2 ± 4.94 | 87.6 ± 3.34 | 81.8 ± 2.25 | 79.3 ± 5.63 | 7.1 ± 1.49 |
| 0.39 | 50.6 ± 27.00 | 28.5 ± 4.60 | 40.5 ± 12.72 | 64.4 ± 16.47 | 62.7 ± 2.84 | 46.6 ± 19.69 | 4.8 ± 4.21 |
| 0.20 | 20.46 ± 8.34 | 22.9 ± 0.40 | 23.6 ± 10.19 | 31.4 ± 11.15 | 21.0 ± 11.6 | 17.5 ± 6.73 | 7.2 ± 0.17 |
| 0.10 | 8.4 ± 2.08 | 12.1 ± 11.32 | 17.8 ± 1.31 | 23.2 ± 6.69 | 15.7 ± 16.13 | 11.1 ± 7.63 | 3.8 ± 5.51 |

TABLE 6

| | SARS-CoV-2 virus 50% | |
|---|---|---|
| | inhibitory ability | |
| Test group | (IC₅₀ value) | |
| Anhydrous ethanol immersion | 1.736 | ug/mL |
| extract of Justicia procumbens | | |
| Justicidin-A | 0.159 | uM |
| Justicidin-B | 0.203 | uM |
| 6′ hydroxyl justicidin-B | 1.060 | uM |
| Remdesivir (uM) - | 10.776 | uM |
| Positive control group | | |
| Lopinavir (uM) - | 12.649 | uM |
| Positive control group | | |
| Chloroquine (uM) - | 8.781 | uM |
| Positive control group | | |

TABLE 7

| | SARS-CoV-2 virus 50% | |
|---|---|---|
| | inhibitory ability | |
| Test group | (IC₅₀ value) | |
| Water extract of Justicia procumbens | >50 | ug/mL |
| 30% ethanol extract of Justicia procumbens | 4.461 | ug/mL |
| 60% ethanol extract of Justicia procumbens | 1.873 | ug/mL |
| 95% ethanol extract of Justicia procumbens | 1.243 | ug/mL |
| 100% ethanol extract of Justicia procumbens | 0.612 | ug/mL |
| Isopropanol extract of Justicia procumbens | 0.409 | ug/mL |
| Methanol extract of Justicia procumbens | 1.034 | ug/mL |
| Butanol extract of Justicia procumbens | 0.465 | ug/mL |
| Acetone extract of Justicia procumbens | 0.308 | ug/mL |
| Dichloromethane extract of | 0.345 | ug/mL |
| Justicia procumbens | | |
| Ethyl acetate extract of Justicia procumbens | 0.429 | ug/mL |
| Lopinavir (uM) - Positive control group | 14.475 | uM |
| Remdesivir (uM) - Positive control group | 12.701 | uM |

TABLE 8

| | Cell viability (%, average ± standard deviation) | | | | | |
|---|---|---|---|---|---|---|
| | Anhydrous | | | | | |
| | ethanol | | | | | |
| | immersion | | | Remdesivir | Lopinavir | Chloroquine |
| | extract of | | | (uM)- | (uM)- | (uM)- |
| Con- | Justicia | | 6′ hydroxyl | Positive | Positive | Positive |
| cent- | procumbens | Justicidin-B | justicidin-B | control | control | control |
| ration | (ug/mL) | (uM) | (uM) | group | group | group |
| 50 | 93.86 ± 0.24 | 78.67 ± 2.53 | 93.38 ± 0.72 | 102.40 ± 4.28 | 74.13 ± 1.69 | 59.69 ± 1.38 |
| 25 | 98.03 ± 6.58 | 83.49 ± 1.22 | 97.80 ± 1.29 | 107.95 ± 0.43 | 77.95 ± 1.50 | 71.29 ± 6.06 |
| 12.5 | 101.80 ± 6.36 | 90.01 ± 1.60 | 101.11 ± 0.36 | 104.4 ± 91.82 | 92.36 ± 2.16 | 83.63 ± 0.73 |
| 6.25 | 105.57 ± 7.31 | 94.60 ± 3.46 | 103.44 ± 1.31 | 98.92 ± 1.05 | 87.57 ± 7.72 | 90.84 ± 3.72 |
| 3.13 | 110.10 ± 3.31 | 98.69 ± 5.68 | 105.46 ± 0.38 | 94.32 ± 6.45 | 89.54 ± 1.11 | 97.41 ± 1.82 |
| 1.56 | 107.98 ± 8.24 | 102.83 ± 5.24 | 107.86 ± 0.60 | 97.46 ± 6.58 | 91.64 ± 6.21 | 89.11 ± 4.21 |
| 0.78 | 108.86 ± 6.82 | 108.21 ± 2.14 | 104.18 ± 3.43 | 92.43 ± 4.42 | 93.08 ± 6.02 | 91.66 ± 2.39 |
| 0.39 | 103.45 ± 7.55 | 107.70 ± 4.88 | 105.17 ± 0.85 | 95.76 ± 6.64 | 88.89 ± 3.07 | 83.14 ± 3.44 |
| 0.20 | 103.75 ± 3.25 | 110.62 ± 0.00 | 104.63 ± 0.44 | 88.32 ± 1.19 | 94.69 ± 5.45 | 87.70 ± 0.37 |
| 0.10 | 106.63 ± 1.14 | 107.08 ± 1.75 | 100.66 ± 0.73 | 98.25 ± 4.51 | 86.74 ± 0.49 | 83.10 ± 6.34 |

TABLE 9

| | Cell viability (%, average ± standard deviation) | | | | | | |
|---|---|---|---|---|---|---|---|
| | | | 30% | 60% | 95% | 100% | Lopinavir |
| | | Water | ethanol | ethanol | ethanol | ethanol | (uM) - |
| Con- | | extract of | extract of | extract of | extract of | extract of | Positive |
| cent- | Justicidin-A | procumbens | procumbens | procumbens | procumbens | procumbens | control |
| ration | (uM) | (ug/mL) | (ug/mL) | (ug/mL) | (ug/mL) | (ug/mL) | group |
| 50 | 98.0 ± 3.04 | 94.5 ± 0.56 | 96.7 ± 0.93 | 92.0 ± 1.94 | 89.0 ± 1.73 | 85.0 ± 3.92 | 68.8 ± 2.30 |
| 25 | 99.5 ± 1.81 | 94.4 ± 0.07 | 96.9 ± 1.80 | 98.1 ± 0.63 | 93.4 ± 4.41 | 89.6 ± 1.83 | 87.0 ± 1.45 |
| 12.5 | 96.5 ± 0.20 | 94.4 ± 2.34 | 101.2 ± 1.89 | 98.8 ± 0.23 | 97.0 ± 1.73 | 93.4 ± 1.00 | 90.5 ± 0.75 |
| 6.25 | 96.9 ± 3.33 | 96.3 ± 2.68 | 101.3 ± 0.17 | 101.9 ± 0.30 | 99.8 ± 2.30 | 96.7 ± 2.43 | 92.0 ± 4.01 |
| 3.13 | 98.4 ± 1.93 | 94.9 ± 0.41 | 100.4 ± 0.66 | 101.0 ± 0.92 | 102.0 ± 0.15 | 98.6 ± 4.03 | 94.2 ± 3.17 |
| 1.56 | 97.4 ± 0.09 | 98.8 ± 2.61 | 98.8 ± 0.68 | 102.0 ± 2.15 | 104.6 ± 0.30 | 101.1 ± 0.44 | 93.3 ± 1.23 |
| 0.78 | 100.1 ± 1.00 | 94.7 ± 2.51 | 98.7 ± 2.00 | 98.4 ± 0.72 | 101.2 ± 1.76 | 100.3 ± 2.83 | 86.6 ± 8.13 |
| 0.39 | 101.3 ± 1.55 | 96.7 ± 2.79 | 99.9 ± 1.79 | 101.9 ± 0.93 | 101.6 ± 1.00 | 1004.3 ± 2.83 | 85.0 ± 13.65 |
| 0.20 | 97.6 ± 0.61 | 92.4 ± 0.25 | 94.2 ± 4.87 | 96.4 ± 0.89 | 95.3 ± 0.86 | 95.6 ± 0.45 | 91.8 ± 2.06 |
| 6.10 | 96.7 ± 2.19 | 95.7 ± 1.07 | 98.6 ± 2.58 | 96.9 ± 2.24 | 99.2 ± 2.46 | 99.0 ± 2.84 | 94.6 ± 1.80 |

TABLE 10

| | SARS-CoV-2 virus 50% | |
|---|---|---|
| | inhibitory ability | |
| Test group | (IC₅₀ value) | |
| Anhydrous ethanol immersion | 1.18 | ug/mL |
| extract of Justicia procumbens | | |
| Justicidin-B | 0.17 | uM |
| 6′ hydroxyl justicidin-B | 0.05 | uM |
| Hydroxy-chloroquine (uM) - | 9.33 | uM |
| Positive control group | | |

As can be understood from above Tables 3 to 10 and FIG. 3, it was confirmed that the anhydrous ethanol extracts of Justicia procumbens prepared by Preparing Examples 1 to 4 and the active ingredients thereof, such as justicidin-A, justicidin-B, and 6′ hydroxyl justicidin-B show a SARS-CoV-2 inhibitory ability remarkably more excellent than that of Remdesivir, which is a positive control group, Lopinavir and Chloroquine, which are main ingredients of Kaletra, when being compared with each other at 50% inhibitory concentration (IC₅₀) values. These test groups showed no particular cytotoxicity up to the highest concentration tested.

In addition, in case of the hydrous ethanol extract and organic solvent extract of Justicia procumbens, which were prepared by Preparing Example 5, it was confirmed that all the hydrous ethanol extracts excluding the water extract and organic solvent extracts show an excellent SARS-CoV-2 inhibitory ability, and all the extracts show at least 80% of cell viability even at 50 ug/mL with regard to cytotoxicity.

### EXAMPLE 3

### Experiment on Comparison of Therapeutic Effects In Vero Cell Lines Infected with SARS-CoV-2

To confirm a SARS-CoV-2 treatment effect of the anhydrous ethanol immersion extract of Justicia procumbens and main ingredients thereof such as justicidin-B and 6′ hydroxyl justicidin-B, the VERO cells were cultured (2×10⁴ cell/well), and then infected with SARS-CoV-2 virus at MOI 0.1 (2×10³ PFU/100 ul) in an incubator at 37° C. for one hour. The infected cells were washed with PBS, and the drugs prepared according to Preparing Examples 1, 3 and 4 were added to the cells for each concentration. And, a culture fluid was obtained after 48 hours, an RNA level present in the SARS-CoV-2 virus particles was measured by qRT-PCR, and comparative analysis was performed with a control group (a viral infected group without drug treatment). To measure the IC₅₀ of the drug, the concentration started at 100 uM and diluted by two steps before use. In this case, the cell viability was confirmed by WST-1 assay.

TABLE 11

| | SARS-CoV-2 virus 50% | |
|---|---|---|
| | inhibitory ability | |
| Test group | (IC₅₀ value) | |
| Anhydrous ethanol immersion | 1.18 | ug/mL |
| extract of Justicia procumbens | | |
| Justicidin-B | 0.17 | uM |
| 6′ hydroxyl justicidin-B | 0.05 | uM |
| Hydroxy-chloroquine (uM) - | 9.33 | uM |
| Positive control group | | |

TABLE 12

| | 50% cell viability | |
|---|---|---|
| | inhibitory concentration | |
| Test group | (CC₅₀ value) | |
| Anhydrous ethanol immersion | 340 | ug/mL |
| extract of Justicia procumbens | | |
| Justicidin-B | >800 | uM |
| 6′ hydroxyl justicidin-B | 314 | uM |
| Hydroxy-chloroquine (uM) - | 160 | uM |
| Positive control group | | |

As can be understood from above Table 11, when the VERO cells previously infected with SARS-CoV-2 virus one hour before are treated with the anhydrous ethanol immersion extract of Justicia procumbens and the active ingredients thereof, such as justicidin-B, and 6′ hydroxyl justicidin-B, it was confirm that this extract and those ingredients shows a SARS-CoV-2 inhibitory ability about 8 to 187 times higher than that of Hydroxychloroquine, a positive control group, when being compared with each other at 50% inhibitory concentration (IC₅₀) values. It was just like the case of the VERO cells being simultaneously treated with the virus and the drug. Base on the results of 50% cell viability concentration in Table 12, the concentration showing cytotoxicity of these test groups was at least about 288 times higher compared to a concentration showing the drug efficacy, thereby it was confirm that the test drug is very safe.

In Formulation Examples 1 to 3 below, the examples of preparing drug medicines, foods, and beverages according to one example of the present invention will be described in detail. In Preparing Example below, the extract of Justicia procumbens may include at least one selected from the methanol, ethanol, isopropanol, butanol, hexane, ethyl acetate, dichloromethane, ether, chloroform, and acetone extracts of Justicia procumbens.

### FORMULATION EXAMPLE 1

### Preparation of Medicament

1-1: Preparation of Powder

100 mg of extract of Justicia procumbens, justicidin-A, justicidin-B, or 6′ hydroxyl justicidin-B

Lactose 100 mg

Talc 10 mg

The above ingredients were mixed and filled into an airtight pack to prepare powder.

1-2 Preparation of Tablet

100 mg of extract of Justicia procumbens, justicidin-A, justicidin-B, or 6′ hydroxyl justicidin-B

Maize starch 100 mg

Lactose 100 mg

Magnesium stearate 2 mg

The above ingredients were mixed and compressed to prepare a tablet according to a conventional method for preparing tablets.

1-3 Preparation of Capsule

100 mg of extract of Justicia procumbens, justicidin-A, justicidin-B, or 6′ hydroxyl justicidin-B

Maize starch 100 mg

Lactose 100 mg

Magnesium stearate 2 mg

The above ingredients were mixed and filled into a gelatin capsule to prepare a tablet according to a conventional method for preparing capsules.

1-4 Preparation of Injection

100 mg of extract of Justicia procumbens, justicidin-B, justicidin-A, or 6′ hydroxyl justicidin-B

Suitable amount of sterilized distilled water for injection

Suitable amount of pH adjusting agent

An injection was prepared with the above content of ingredients per ampoule (2 ml) according to a conventional method for preparing injections.

1-5 Preparation of Liquid Medicine

100 mg of extract of Justicia procumbens, justicidin-A, justicidin-B, or 6′ hydroxyl justicidin-B

Sugar 20 g

Isomerized glucose syrup 20 g

Suitable amount of lemony flavor

Distilled water was added to adjust the total amount to 1.00 ml. The above ingredients were mixed according to a conventional method for preparing liquid medicines, and then filled into a brown bottle, and sterilized to prepare a liquid medicine.

1-6 Preparation of Inhalant

100 mg of extract of Justicia procumbens, justicidin-A, justicidin-B, or 6′ hydroxyl justicidin-B

1,1,1,2-tetrafluoroethane 15 g

Anhydrous ethanol 1.5 g

Citric acid (anhydride) 0.05 mg

Polyethylene glycol 500 mg

The above ingredients were mixed according to a conventional method for preparing inhalants, and then filled into a container.

### FORMULATION EXAMPLE 2

### Preparation of Food

100 mg of extract of Justicia procumbens, justicidin-A, justicidin-B, or 6′ hydroxyl justicidin-B

Suitable amount of vitamin mixture

Vitamin A acetate 70 μg

Vitamin E 1.0 mg

Vitamin B1 0.13 mg

Vitamin B2 0.15 mg

Vitamin B6 0.5 mg

Vitamin B12 0.2 μg

Vitamin C 10 mg

Biotin 10 μg

Nicotinic acid amide 1.7 mg

Folic acid 50 μg

Calcium pantothenate 0.5 mg

Suitable amount of mineral mixture

Ferrous sulfate 1.75 mg

Zinc oxide 0.82 mg

Potassium phosphate monobasic 15 mg

Calcium phosphate dibasic 55 mg

Potassium citrate 90 mg

Calcium carbonate 100 mg

Magnesium chloride 24.8 mg

As for a composition ratio of the above vitamin and mineral mixtures, ingredients suitable for health functional foods were mixed according to a preferred example, but a mixing ratio thereof may be arbitrarily modified, and the above ingredients may be mixed according to a conventional method for preparing health functional foods, and then may be used in preparing a health functional food composition (e.g., nutritional candy, etc.) according to a conventional method.

### FORMULATION EXAMPLE 3

### Preparation of Beverage

100 mg of extract of Justicia procumbens, justicidin-A, justicidin-B, or 6′ hydroxyl justicidin-B

Citric acid 1000 mg

Oligosaccharide 100 g

Plum concentrate 2 g

Taurine 1 g

Distilled water was added to adjust the total amount to 900 ml.

The above ingredients were mixed according to a conventional method for preparing health functional beverages, and then stirred and heated at 85° C. for about one hour, after which the resulting solution was filtered and collected into a sterilized 2 [symbol] ontainer, sealed and sterilized, and then stored in a refrigerator. After that, the resulting product was used in preparing the health functional beverage composition of the present invention.

As for the above composition ratio, the ingredients relatively suitable for a preferred beverage were mixed according to a preferred example, but a mixing ratio thereof may be arbitrarily modified according to regional and ethnic preferences such as a class of demand, a country of demand, an intended use, etc.

1. A method for preventing, ameliorating or treating diseases caused by SARS-CoV-2, comprising administering a pharmaceutical composition comprising an organic solvent extract of Justicia procumbens into a subject in need thereof.

2. The method according to claim 1, wherein the organic solvent is an alcohol having 1 or more and 4 or less carbon atoms.

3. The method according to claim 1, wherein the organic solvent is at least one selected from methanol, ethanol, isopropanol, butanol, hexane, ethyl acetate, dichloromethane, ether, chloroform, and acetone.

4. The method according to claim 1, wherein the organic solvent is anhydrous ethanol.

5. The method according to claim 1, wherein the disease caused by said SARS-CoV-2 comprises coronavirus disease-19.

6. The method according to claim 5, wherein a symptom of the coronavirus disease-19 is at least one of fever, feebleness, cough, dyspnea, pneumonia, phlegm, sore throat, headache, hemoptysis, nausea, and diarrhea.

7. The method according to claim 1, wherein the disease caused by said SARS-CoV-2 is a respiratory disease.

8. The method according to claim 7, wherein the respiratory disease is pneumonia.

9. A method for preventing, ameliorating or treating diseases caused by SARS-CoV-2, comprising administering a pharmaceutical composition comprising justicidin-A into a subject in need thereof.

10. A method for preventing, ameliorating or treating diseases caused by SARS-CoV-2, comprising administering a pharmaceutical composition comprising justicidin-B into a subject in need thereof.

11. A method for preventing, ameliorating or treating diseases caused by SARS-CoV-2, comprising administering a pharmaceutical composition comprising 6′ hydroxyl justicidin-B into a subject in need thereof.

12. A method for preventing, ameliorating or treating diseases caused by SARS-CoV-2, comprising administering a pharmaceutical composition comprising an extract of Justicia procumbens into a subject in need thereof.

13. An anti-viral method against SARS-CoV-2, comprising administering a composition comprising justicidin-A into a subject in need thereof.

14. An anti-viral method against SARS-CoV-2, comprising administering a composition comprising justicidin-B into a subject in need thereof.

15. An anti-viral method against SARS-CoV-2, comprising administering a composition comprising 6′ hydroxyl Justicidin-B into a subject in need thereof.

16-35. (canceled)
